(19) United States
(12) Patent Application Publication
(10) Pub. No.: US 2006/0057228 A1
(43) Pub. Date: Mar. 16, 2006

(54) Weight loss composition

(75) Inventors: Qun Yi Zheng, Wayne, NJ (US); Bo Lin Zheng, Aurora, CO (US); Kan He, River Edge, NJ (US); Bao Liang Cui, Shrewsbury, MA (US); Naisheng Bai, Highland, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pure World Botanicals, Inc., South Hackensack, NJ (US)

(21) Appl. No.: 11/178,239

(22) Filed: Jul. 8, 2005

Related U.S. Application Data
(60) Provisional application No. 60/586,851, filed on Jul. 9, 2004.

(51) Int. Cl. A61K 36/82; A61K 36/75; A61K 36/484; A61K 31/375; A61K 31/198

(52) U.S. Cl. 424/725; 424/729; 424/757; 514/474; 514/561

(57) ABSTRACT

The present invention provides a dietary supplement and methods of using the same to promote body weight reduction and/or loss of body fat in a subject, and for the management of obesity. The supplement can further be used to reduce one or more metabolic parameters in a subject, such as blood glucose levels, blood triglyceride levels, and blood cholesterol levels.

This application claims the benefit of U.S. provisional application No. 60/586,851, filed Jul. 9, 2004, which is hereby incorporated by reference in its entirety.

### BACKGROUND OF THE INVENTION

1. Field of the Invention

The present invention provides a dietary supplement and methods of using the same to promote body weight reduction and/or fat loss and for the management of obesity. The supplement can further be used to reduce one or more metabolic parameters in a subject, such as blood glucose levels, blood triglyceride levels, and blood cholesterol levels.

2. Description of the State of Art

Weight control is a concern of human beings. More importantly, excessive accumulation of body fat (i.e. obesity) can be dangerous and has been linked to health problems such as type II diabetes, hypertension, hyperlipidemia, coronary heart disease, stroke, breast and colon cancer, sleep apnea, gallbladder disease, gastroesophogeal reflux disease, fatty liver, gout, thromboembolism. Obesity is one of the main sectors of cardiovascular diseases. The levels of cholesterol, blood pressure, blood sugar and uric acid in obese people are usually higher than those of persons of normal weight. The morbidity from coronary heart disease among the overweight people is increased as well. Among the people aged 40-50, mortality will rise about 1% when body weight increases by 0.5 kg and the death rate will increase 74% when body weight exceeds 25% of the standard. Despite increased awareness of these health risks among Americans, the prevalence of obesity in the United States has more than doubled since the turn of the century.

Determinants of obesity include social factors, psychological factors, genetic factors, developmental factors, and decreased physical activity. Some components of a comprehensive weight loss program include medical assessment, behavior and dietary modification, nutrition education, cognitive restructuring, increased physical activity, and long term follow-up.

Up to now, there has been no effective and safe medicine to cure physiological obesity. Current weight loss medicines can be divided into two groups depending on their mechanisms of action: those affecting the central nervous system, and those that do not affect the central nervous system. The first type can reduce weight by affecting certain neurotransmitters to decrease appetite, but its side effects include a change in blood pressure or heart rate when taken for a short term. The second type is a lipase depressant. It can affect weight by reducing the absorption rate of fat. However, its side effects include reducing the absorption of some important liposoluble vitamins, and therefore it may break down the balance of one's nutrition. Other current weight loss medicines or health foods are not very good due to their minimal effect or unwanted side effects.

When fat stored in adipose tissue is going to be used as an energy source, lipase enzymes hydrolyze triglycerides into glycerol and free fatty acids in a process called lipolysis (the breakdown of fat). These molecules (primarily the free fatty acids) serve as blood-borne energy carriers that can be used by the liver, skeletal muscles, and other organs for aerobic respiration.

Insulin is secreted when there is high sugar content; insulin secretion allows for fat storage. High insulin levels trigger the hypothalamus to send hunger signals, which sets off a craving for carbohydrates; this leads one to eat more, which leads to more insulin. Excess carbohydrates are converted into glucose, and then stored as fat.

Glucose triggers a rise in insulin. Insulin acts to lower blood glucose levels, regulating those levels through several actions, including lipogenesis (conversion of carbohydrate and protein into fat). When the insulin levels are high lipolysis is blocked and fat is stored. The body must initiate lipolysis to supply the cellular energy source ATP, which is necessary for muscle contraction (energy). When insulin levels are reduced, fat is burned to provide energy.

Fenugreek is a food and a spice containing 4-hydroxyisolucine, isoflavone, saponion, stolon, trigonelline, diosgenin and polysaccharides. It has been used for centuries by practitioners of Ayurvedic and traditional Chinese medicine. Recent studies have investigated the blood cholesterol-lowering and blood glucose-lowering properties of fenugreek seeds, both in normal subjects and in those with diabetes. Significant reductions in total cholesterol, LDL cholesterol, and triglycerides levels, but not HDL cholesterol levels, have been observed in non-insulin-dependent diabetics consuming 25 grams of fenugreek per day. With only five grams of fenugreek per day, fasting and postprandial blood glucose levels were significantly reduced in those persons with diabetes. Currently fenugreek is recognized as a useful botanical aid in the treatment of persons with diabetes. For diabetics who are insulin dependent, a higher level of fenugreek is needed to significantly reduce fasting blood glucose levels as well as reduce LDL cholesterol and triglycerides levels. Recently it was observed that consumption of 12 to 18 grams of germinated fenugreek seed powder for 30 days resulted in a significant reduction in blood cholesterol and LD cholesterol levels, but not HDL cholesterol levels, in 20 adults who had normal cholesterol levels initially.

Banaba is a medicinal plant that grows in India, Southeast Asia and the Philippines. Traditional uses include brewing tea from the leaves as a treatment for diabetes and hyperglycemia. The hypoglycemic (blood sugar lowering) effect of banaba leaf extract is similar to that of insulin. The blood sugar regulating properties of banaba have been demonstrated in animal and human studies. For example, in humans with type II diabetes, banaba extract, at a dose of 16-48 mg per day for 4-8 weeks, has been shown to be effective in reducing blood sugar levels (5% to 30% reduction) and maintaining tighter control of blood sugar fluctuations. An observed side-effect of tighter control of blood sugar and insulin levels was a tendency of banaba to promote weight loss (an average of 2-4 pounds per month) without significant dietary alterations. It was suggested that modulation of glucose and insulin levels reduces total caloric intake somewhat and encourages moderate weight loss.

Yerba mate is an evergreen member of the holly family that grows wild in South America. The primary active chemical constituency of yerba mate comprises xanthine alkaloids (caffeine, theobromine, and teophylline), saponins, chlorogenic acid, caffeic acid, and caffeoylquinic acids, as well as triterpenoid saponins. Yerba mate tea is said to stimulate the nervous and muscular systems and is used for digestive problems, renal colic, neurasthenia, depression, fatigue, and obesity.

Green tea leaf (Camellia sinesis) extract stimulates brown adipose tissue thermogenesis to an extent that is much greater than can be attributed to its caffeine content per se. It has been proposed that the thermogenic properties of green tea reside primarily in an interaction between its high content in catechin-polyphenols and caffeine with sympathetically released noradrenaline (Dulloo et al., Int. J. of Obesity, 24:252-258 (2000)).

Huzhang is an east Asian species (Polygonum cuspidatum), belonging to the Polygonaceae family. Huzhang has been used to treat acute microbial infections, viral hepatitis, chemo- or radiation-induced leukocytopenia, atherosclerosis, and hyperlipidemina.

Goat's rue (Galega officinalis) is another traditional remedy for diabetes, and it has been shown to have hypoglycemic activity in humans.

There remains a need for a safe and effective composition for promoting weight loss and/or loss of body fat in subjects such as humans.

### BRIEF SUMMARY OF THE INVENTION

The present invention provides a dietary supplement and methods of using the same for promoting weight loss and/or loss of body fat in a subject, and for the management of obesity. The compositions of this invention comprises a mixture of 20% 4-hydroxyl isoleucine (purified from Fenugreek), Banaba, Huzhang, Yerba mate, and Green tea, and is intended for administration in any type or form. In another embodiment, the dietary supplement of this invention comprises a mixture of Goat's rue, Banaba extract, Huzhang extract, Yerba mate, and Green tea extract. The compositions may further comprise goat's rue. A representative formula for a composition of this invention is as follows:

| | Ratio in the |
|---|---|
| Components | formula |
| 20% 4-hydroxyisolucine | 6-14% |
| or Goat's rue extract | |
| Yerba mate extract | 34-66% |
| Banaba extract | 16-21% |
| Green tea extract | 3-14% |
| Huzhang extract | 3-14% |
| Vitamin C | 0-0.7% |
| Licorice extract | 0-5% |

The present invention further provides methods for using the compositions of this invention for promoting weight loss and/or body fat loss, and for the treatment of obesity. The formulations of this invention are further useful for reducing at least one metabolic parameter in a subject, such as blood glucose levels, blood triglyceride levels, and blood cholesterol levels.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF DESCRIPTION OF THE DRAWINGS

The accompanying drawings, which are incorporated herein and form a part of the specification, illustrate non-limiting embodiments of the present invention, and together with the description, serve to explain the principles of the invention.

In the Figures:

FIGS. 1A and 1B show the changes in body weight and body fat index, respectively, in mice fed a composition of this invention having Formula A, a high fat diet with thyroid, a high fat diet, or a normal diet.

FIGS. 2A, 2B, and 2C show the changes in blood glucose levels, triglyceride levels and cholesterol levels, respectively, in mice fed a composition of this invention having Formula A, a high fat diet with thyroid, a high fat diet, or a normal diet.

FIGS. 3A and 3B show the differences in body weight and food consumption, respectively, over a 30 day period in mice feed either a composition of this invention having Formula A or a control diet.

FIG. 4 shows the differences in body fat on day 1 (white column) and day 30 (black column) for 23 test subjects after daily administration of a composition comprising Formula B of this invention.

FIG. 5 shows the differences in body weight on day 1 (white columns) and day 30 (black columns) for 23 test subjects after daily administration of a composition comprising Formula B of this invention.

FIG. 6 shows the relative percent change in body weight (striped columns) and body fat (solid columns) for 23 test subjects after daily administration of a composition comprising Formula B of this invention.

FIGS. 7A, 7B, 7C and 7D show the chemical structures of Banasulin, Lagerstroemin, Casuarinin, and 3-O-methylellegic acid, respectively.

### DETAILED DESCRIPTION OF THE INVENTION

The present invention provides a dietary supplement for body weight reduction and for the management of obesity. The dietary supplement of this invention comprises a mixture of 20% 4-hydroxyl isoleucine (purified from Fenugreek), Banaba, Huzhang, Yerba mate, and Green tea, and is intended for administration in any type or form. In another embodiment, the dietary supplement of this invention comprises a mixture of Goat's rue, Banaba, Huzhang, Yerba mate, and Green tea.

A representative Formula A for a composition of this invention is shown in Table 1:

TABLE 1

| | amt. (mg) in |
|---|---|
| Components | Formula A |
| 20% 4-Hydroxyisolucine | 20-200 |
| Yerba mate extract | 200-500 |
| Banaba extract | 50-300 |
| Green tea extract | 10-200 |
| Huzhang extract | 10-200 |
| Vitamin C | 1-10 |
| Licorice extract | 10-50 |

A representative Formula B for a dietary supplement of this invention is shown in Table 2:

TABLE 2

| | amt. (mg) in |
|---|---|
| Components | Formula B |
| Goat's rue extract | 20-200 |
| Yerba mate extract | 200-500 |
| Banaba extract | 50-300 |
| Green tea extract | 10-200 |
| Huzhang extract | 10-200 |
| Vitamin C | 1-10 |
| Licorice extract | 10-50 |

The compositions of this invention, which comprise a combinative extract of herbs, are prepared using food graded ethanol and water as solvent via a counter current extraction and purification processes described in U.S. Pat. No. 6,682,697, which is incorporated herein by reference. The herbal extracts used in the present invention can be derived from any suitable source. In brief, either the individual plant or the combined components such as those shown in Table 1 and Table 2 were extracted with 10-85% ethanol at room temperature to 75° C. by counter-current extraction technique, chromatography purification and then spray dried to a powder form. The powder can then be formulated as appropriate for the desired form of administration.

A “subject” includes both humans and other animals (particularly mammals) that receive either prophylactic or therapeutic treatment according to this invention.

Pharmaceutical formulations of the present invention can contain as an active ingredients from about 0.5 to about 95.0 wt % of an herbal mixture of this invention. The formulations can be obtained by mixing the herbal mixture with one or more excipients, including, but not limited to agglutinants, disintegrators, lubricants, sliders or fillers. For example, specific excipients include, but are not limited to, lactose, corn starch, saccharose, magnesium stearate, microcrystalline cellulose, sodium croscarmellose gelatin, cellulose acetophtalate, titanium dioxide, special talc for tablets and polyethylene glycol. Other suitable excipients are well known in the art. The formulation may further comprise from 0% to about 65% by weight of pharmaceutically acceptable formulation aids, including but not limited to diluents, stabilizers, binders, buffers, lubricants, coating agents, preservatives, emulsifiers and/or suspension agents.

In some embodiments, a pharmaceutical formulation of this invention is administered in one dosing of a single formulation, and in other embodiments a dietary supplement of this invention is administered in multiple dosings of a single formulation.

The amount of a the herbal mixture in the formulations of this invention that is combined with one or more excipients to produce a single dosage form will necessarily vary depending upon the host treated and the particular route of administration. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In order to use the compositions of this invention, the herbal mixtures are formulated in accordance with standard pharmaceutical practice as a pharmaceutical composition as discussed above. According to this aspect of the invention there is provided a pharmaceutical formulation comprising an herbal mixture of this invention, wherein the herbal mixture is present in an amount effective for promoting body weight loss and/or body fat loss, or for managing obesity in a subject. According to another aspect of the invention there is provided a pharmaceutical formulation comprising an herbal mixture of this invention, wherein the herbal mixture is present in an amount effective to reduced one or more metabolic parameters in a subject.

The formulations of the present invention can be administered to a subject by any available and effective delivery system including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and in a vehicle as desired, such as a depot or a controlled release formulation. Preferably, the formulations are administered orally.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), or for parenteral administration (for example as a sterile aqueous or oily solution for intravenous, subcutaneous, or intramuscular dosing or as a suppository for rectal dosing), or as vehicles such as a depot or other controlled release formulation.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art. Therapeutic formulations suitable for oral administration, e.g., tablets and pills, may be obtained by compression or molding, optionally with one or more accessory ingredients. Compressed tablets may be prepared by compressing the herbal mixture in a suitable apparatus into tablets having a suitable size. Prior to the mixing, the herbal mixture may be mixed with a binder, a lubricant, an inert diluent and/or a disintegrating agent.

Formulations for oral use may be in the form of hard gelatin capsules in which an composition of this invention is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the herbal mixture in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the herbal mixture in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain an herbal mixture of this invention together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, wherein the herbal mixture may be formulated according to known procedures using one or more of the appropriate dispersing or wetting agents and suspending agents, which have been described herein. A sterile injectable preparation may also be a sterile injectable solution or suspension in a non-toxic parenterally-acceptable diluent or solvent, for example a solution in 1,3-butanediol.

Suppository formulations may be prepared by mixing the herbal mixture with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating the herbal mixture with a conventional, topically acceptable, vehicle or diluent using conventional procedures well known in the art.

In yet another embodiment, the therapeutic compound can be delivered in a controlled release system. In one embodiment, a pump may be used (Sefton, CRC Crit. Ref. Biomed. Eng. 14:201 (1987); Buchwald et al., Surgery, 88:507 (1980); Saudek et al., N. Engl. J. Med., (10) 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61 (1983); see also Levy et al., Science 228:190 (1985); During et al., Ann. Neurol, 25:351 (1989); Howard et al., J. Neurosurg. 71:105 (1989)). Other controlled release systems are discussed in the review by Langer (Science 249:1527-1533 (1990)).

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The invention is further illustrated by the following non-limiting examples. All scientific and technical terms have the meanings as understood by one with ordinary skill in the art. The specific examples which follow illustrate the methods in which the compositions of the present invention may be prepared and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLES

### Example 1

### Efficacy of Formula A in Mice

This example describes the effect of a formulation of this invention on the body weight, parametrical fat pad and retro-peritonael fat pad weight of female SCR mice. In order to evaluate the effect of the formulation, a 4-week controlled study at different doses on female SCR mice was performed. A total of 150 female SCR mice, weighing 18-21 grams, were randomly divided into 12 groups with 15 mice in each group. Nine of the groups were fed a high fat diet mixed with Formulation A (Table 1) comprising 4-hydroxyisolucine powdered extract 20%, yerba mate, banaba extract, green tea extract, huzhang extract, vitamin C (0.3-0.7%, preferably), and licorice extract (3-5%, preferably) in various ratios and at different doses. The remaining three groups were assigned as controls: Group 1 was fed a high fat diet mixed with a thyroid slice, group 2 was fed a high fat diet, and group 3 was fed a normal diet. The change in body weight, diet and water consumption was measured every three days. After a 4-week period, blood samples were collected without anesthesia to measure the concentration of glucose, triglyceride, and cholesterol. The mice were then killed and the parametrial fat pad, retro-peritoneal fat pad, and liver were weighed.

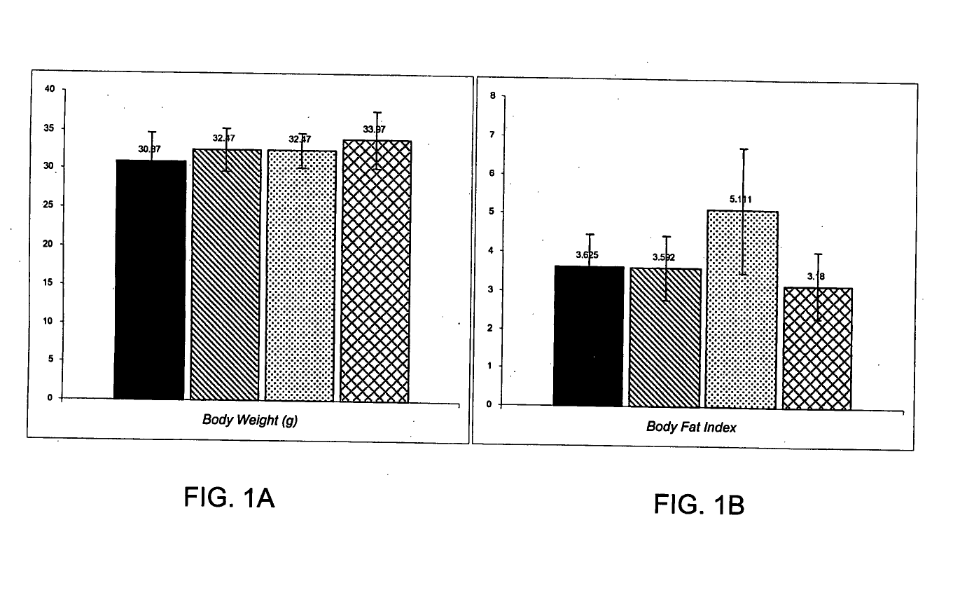

FIGS. 1A and 1B show the changes in body weight and body fat index, respectively, in mice fed a composition of this invention having Formula A, a high fat diet with thyroid, a high fat diet, or a normal diet. As shown in FIGS. 1A and 1B, the highest body weight and body fat was observed in the group fed the high fat diet (group 2) when compared with other groups. The mice in the negative control group were also fed a high fat diet but thyroid slice was added into the diet. Due to the calorigenic and metabolic effects of thyroid, lower fat levels were observed in this group. The major finding in this study was that the formulation of this invention reduces more body fat in female SCR mice as indicated by a significantly larger decrease in body fat index than body weight.

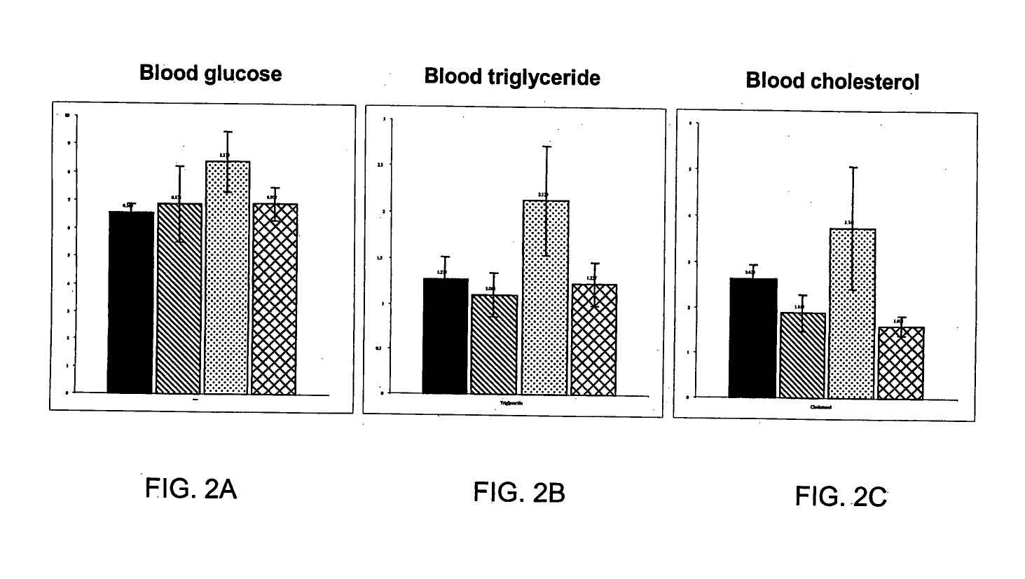

Any discussion of weight loss should consider other metabolic consequences of obesity. For instance, obesity, hyperinsulinism and diabetes are closely related. A high blood glucose level accelerates lipogenesis and fat accumulation through hypersecretion of insulin and insulin requirement. High fat but low insulin response in obese people results in increased lipase activity which increases the free fatty acids in the circulatory system. These fatty acids are the precursors for triacylglycerol and cholesterol synthesis. The compositions of this invention are formulated to have multiple activities that work on the complicated biological system, as shown in FIGS. 2A, 2B, and 2C.

FIGS. 2A, 2B, and 2C show the changes in blood glucose levels, triglyceride levels and cholesterol levels, respectively, in mice fed a composition of this invention having Formula A, a high fat diet with thyroid, a high fat diet, or a normal diet. The mice fed the high fat diet had the highest blood glucose, triglyceride and cholesterol levels when compared with the other groups. The group fed Formula A of this invention had lower blood glucose, triglyceride and cholesterol levels.

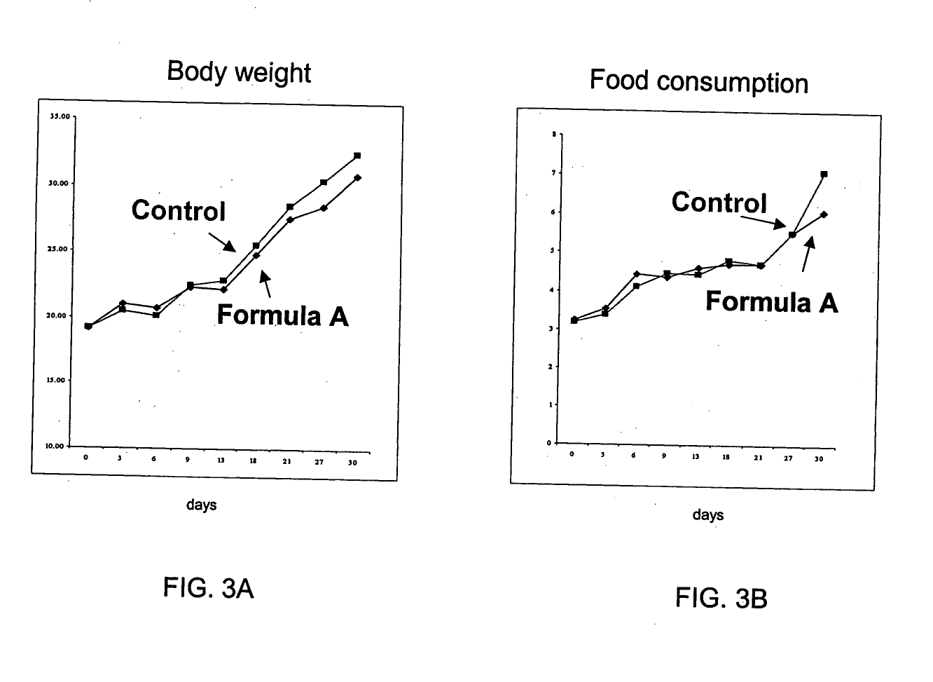

FIGS. 3A and 3B show comparisons of body weight and food consumption, respectively, in mice feed either a control diet or Formula A of this invention. As shown, no difference in food or water consumption was observed in the mice fed Formula A of this invention and weight loss was maintained with increased food consumption, indicating that the mechanism of action of Formula A does not cause appetite suppression.

In conclusion, the animal trial results demonstrated that (1) the group fed Formula A had lower body weight and less body fat gains than the control group; (2) the Formula A group experienced more fat loss than weight loss; and (3) metabolic parameters such as blood glucose, triglycerides and cholesterol levels were lower in the Formula A group than the other groups.

### Example 2

### Efficacy of Formula A and B in Humans

In order to evaluate the effect of the compositions of this invention in humans, a 4-week randomized, double-blind placebo controlled study on humans was performed.

Preparation of herbal mixture: the herbal mixture (Formula A and B; Table 1-2) in this study was provided by Pure World Botanicals, Inc. in the United States through a proprietary cGMP process disclosed in U.S. Pat. No. 6,552,206, U.S. Pat. No. 6,428,824, U.S. Pat. No. 6,361,815 and U.S. Pat. No. 6,267,995, which are incorporated herein by reference.

Capsule: Formula A and B capsules contained 500 mg of Formula A and B (see Table 1 and 2) comprising 20-200 mg of 20% 4-hydroxyisolucine (Table 1) or goat's rue (Table 2), 50-300 mg of banaba extract, 200-500 mg of yerba mate extract, 10-200 mg huzhang extract, 10-200 mg green tea extract, 1-10 mg of Vitamin C, and 10-50 mg of licorice extract.

Subjects were required to be between 25 and 55 years of age with body mass index (BMI)>29 and <35 kg/m² and to have bodyweight stable for at least 3 months prior to evaluation. Excluded were those who were pregnant or had been nursing within the previous year, and subjects having diabetes, thyroid, kidney, liver or heart disease, cancer, anemia, high blood pressure, and those using medication other than contraceptives, hormone replacement therapy, or specific allergy preparations.

Selected for the preliminary trial were 10 males and 13 females with a BMI ranging from 24 to 43 and an average BMI of 31.3, and the subjects were placed wither in the active treatment group or the placebo group. At the beginning of the study, the subjects were evaluated for baseline measurements.

The daily dosage in this study was 4 capsules/person/day. Subjects were instructed to take two capsules filled with Formula A and B (500 mg per capsule) or placebo twice daily along with meals and eight ounces of water. The subjects returned at day 14 and at day 30 to be weighed and measured.

All subjects were advised to limited intake of dietary fat to 30% of calories and to exercise moderately (e.g. walking 30 min/day, three times a week). Handouts on good eating habits and a progressive walking/exercise program were provided. Subjects were directed to take two capsules, 30 minutes before each meal, twice a day, and return the unused, which were counted to determine compliance.

Questionnaires and Measurements:
a. Bowel habit, level of satiety.
b. Medical and nutrition history.
c. Physical examination: body temperature, blood pressure, heart rate, and pulse.
d. Blood and urine studies included serum lipids (cholesterol and triglycerides), liver tests, thyroid hormone, glucose, standard electrolytes, ALT, AST and GGT, a complete blood count, and urine analysis.
e. Electro-cardiogram (ECG): evaluated for four intervals (R-R, P-R, QTc, QRS), QRS amplitude and cardiac rhythm.
f. Body composition measurements: body weight and height were measured to the nearest 0.1 kg and 0.5 cm. Waist and hip circumferences were measured by trained observers.
g. Total body fat was assessed by air displacement plethysmography (Bodpod Body Composition System, Life Measurement Instruments, Concord, Calif.) with an error of 0.1% fat.

Self-Reported Symptoms:

a. Cardiovascular symptoms: chest pain, loud heart beat, palpitations, elevated blood pressure, irregular heart beat, multifocal ventricular event, ventricular event.

b. Gastrointestinal symptoms: dry mouth, heartburn, upset stomach, constipation, diarrhea.

c. Difficulty concentrating.

d. Dizziness.

e. Insomnia.

f. Anxiety.

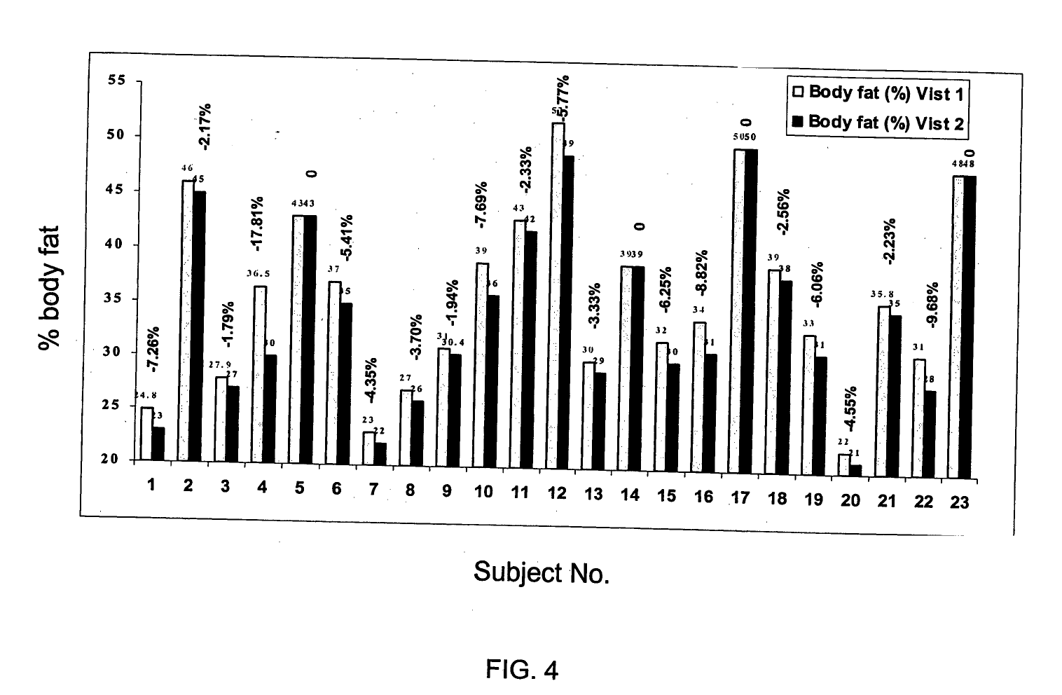

The results of the human trials are shown in FIGS. 4-6. FIG. 4 shows the differences in body fat on day 1 (white column) and day 30 (black column) for the 23 test subjects. The percent body fat lost by 19 of the 23 subjects ranged from 1% to 6.5%. The average percent body fat reduction after taking the Formula A and B tablets during the 4 week span was 1.8%. Four of the subjects maintained their body fat, while no subjects gained body fat.

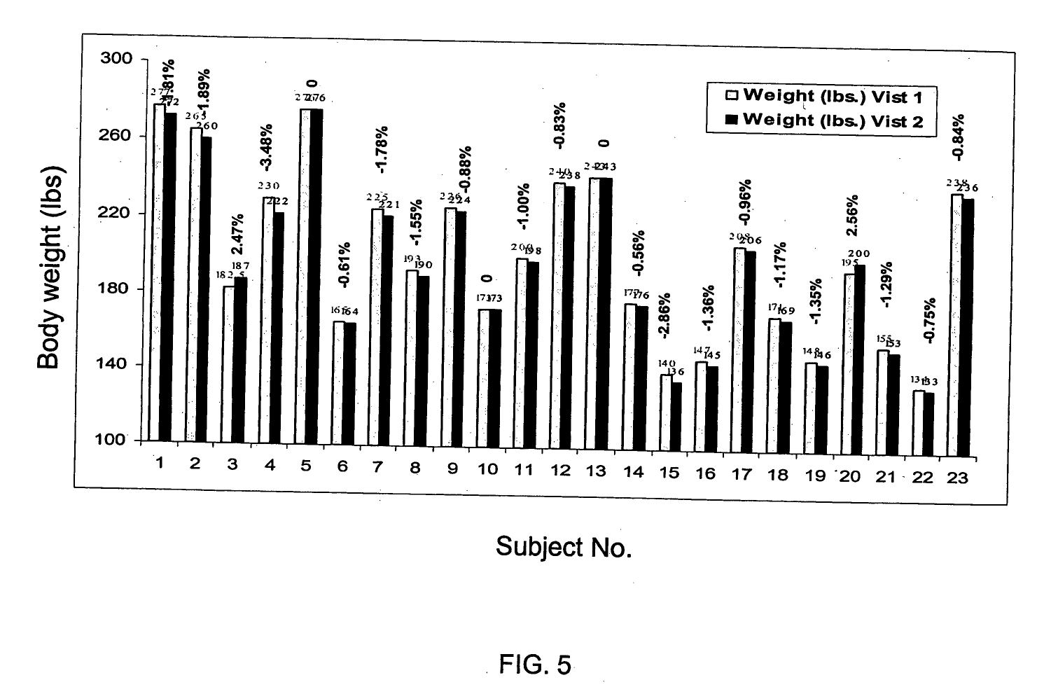

FIG. 5 shows the differences in body weight on day 1 (white columns) and day 30 (black columns) for 23 test subjects. The weight loss ranged from 1 to 8 pounds after taking the Formula A and B tablets. An average loss of 2.7 pounds was noted in 18 of the 23 subjects taking the Formula B tablets after 4 weeks. Two of the subjects gained weight, and three maintained their weight.

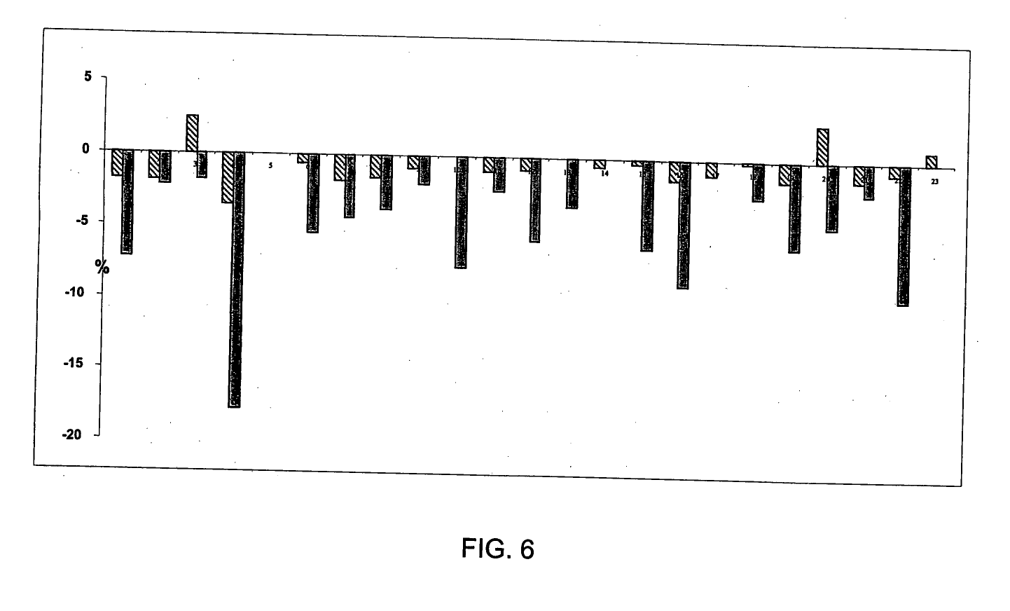

FIG. 6 shows the relative percent change in body weight (striped columns) and body fat (solid columns) for 23 test subjects. The maximum percent body weight reduction was less than 5%, while the maximum body fat reduction was around 17%. Even subjects who gained weight during the 4-week period showed a significant reduction in body fat.

An average weight loss of 4.4 lbs was found in males and 2.7 lbs in females. An average body fat loss of 1.8% was found in both males and females. Therefore, it has been shown that Formula A and B effectively promote weight and fat loss in human trials.

### Example 3

### Efficacy of Banaba Extract and Yerba Extract in Mice

Materials

Banaba extract and yerba extract which were made as described below. Each herb was extracted separately using a food grade ethanol and water system. The alcoholic extracts were concentrated to remove alcohol under vacuum and the residue was further dried to make powdered extracts. Yerba mate leaf extract (ilex paraguariensis) was standardized to 2% caffeine, 0.2% theobromine, and 3% chlorogenic acid contents. The banaba extract was made from the banaba leaf (lagerstroemia speciosa) being extracted with hot water and was standardized containing 2-3% banasulin.

Female ICR mice (Shanghai Seapaul-Beakai Experimental Animal Inc., Grade I) were housed individually in plastic cages in an air-conditioned room (23-25° C.) and fed with water ad libitum and a basal diet. The testing article (Banaba extract and Yerba mate extract) was either dissolved in DI-water or suspended in 50% of ethanol/water solution. Each testing article was administered a daily dose (Table) by gavage to experimental and control groups, respectively. A total of 75 female SCR mice weighing 18-21 g were used for this study. All mice were randomly divided into 5 groups with 15 mice in each group. Two groups were fed a high fat diet mixed with Banaba extract and Yerba mate extract, respectively, for 4 weeks. Three control groups were studied. The negative control group was fed a high fat diet mixed with thyroid slice at a level of 0.04 g/kg, a positive control group was fed a high fat diet, and a normal control group was fed a normal diet. The high fat diet was composed of (in grams per 100 g of diet), 20 g of animal oil, 15 g of refined milk, 0.2 g of methylthio-midin, and 64.8 g of normal diet. Body weight changes, diet and water consumption were measured every 3 days. After one month, blood samples were collected without anesthesia for the measurement of the concentration of glucose, triglyceride, and cholesterol. The mice were then killed and the parametrial fat pad, retro-peritoneal fat pad, and liver were weighed.

The concentration of serum glucose, triglyceride and cholesterol were measured using clinical-analysis kits (Shanghai Biochemical Agent, Inc.). Lee's index was calculated using formula of √ (weight (g)×10³)/body length (cm). The larger the Lee's index is, the fatter the mouse. Body fat index was calculated using the formula of body fat (g)/body weight (g)×100%. The larger the body fat index is, the higher the body fat will be. Results are listed in Table 3.

TABLE 3
Formulation C dose and results on weights of body, fat,
and levels of glucose, triglyceride and cholesterol

| Group | Banaba extract | Yerba mate extract | Thyroid group | Positive control | Normal control |
|---|---|---|---|---|---|
| Dose | 12 | 1.2 | 12 | 12 | 12 |
| (g/kg/day) | | | | | |
| Wt (g) after | 27.57 ± 3.25 | 33.4 ± 3.22 | 32.47 ± 2.83 | 32.47 ± 2.22 | 33.97 ± 3.71 |
| 30 d | | | | | |
| Lee's index | 1.309 ± 0.024*** | 1.377 ± 0.021*** | 1.391 ± 0.037 | 1.409 ± 0.021 | 1.393 ± 0.017* |
| Body fat | 4.074 ± 0.837* | 3.719 ± 0.802* | 3.592 ± 0.827** | 5.111 ± 1.616 | 3.18 ± 0.86*** |
| Index | | | | | |
| Glucose | 6.846 ± 0.969*** | 7.493 ± 0.85 | 6.871 ± 1.334** | 8.375 ± 1.077 | 6.902 ± 0.574*** |
| Triglyceride | 1.556 ± 0.756** | 1.363 ± 0.150*** | 1.098 ± 0.242*** | 2.129 ± 0.59 | 1.227 ± 0.236*** |
| Cholesterol | 3.005 ± 0.487* | 3.271 ± 0.523 | 1.889 ± 0.405*** | 3.741 ± 1.328 | 1.601 ± 0.213*** |

Significant different from positive control, ***P < 0.001. **P < 0.05. *P < 0.01.

### Example 4

### Inhibitory Effect of Banaba Extracts, and Compounds Isolated from Banaba on Undifferentiated 3T3-L1 Cell

The major component of weight gain is the increase and accumulation of fat through adipogenesis process. Adipogenesis is characterized by increase of both fat cell number and fat cell size. The weight loss composition of the present invention, banaba extracts, and some active components which were isolated from banaba have shown strong activity through an inhibitory activity on adipocyte differentiation to block fat cell synthesis to achieve the effect on bodyweight control and body fat loss. The following is the list of the results of anti-adipogenesis assay (Table 4).

TABLE 4
Inhibitory activities of Banaba extracts and some
other components on undifferentiated 3T3-L1 cell

| | Percent of inhibitory | | | |
|---|---|---|---|---|
| | Concentration level (%, w/w) | | | |
| Components | 0.004% | 0.002% | 0.05% | 0.10% |
| Formulation A or B | 45% | 83% | 91% | |
| Banaba extract-1 | 91 | | | |
| Banaba extract-2 | 92% | 100% | 100% | |
| Banaba extract-3 | 90% | 97% | | |
| Banaba extract-4 | 74% | 96% | 99% | 100% |
| Foenugreek PE 20% | 54% | 86% | 87% | 98% |
| Yerba mate extract | 97% | 97% | | |
| Banasulin | 79% | 96% | 99% | |
| Lagerstroemin | 100% | 100% | 100% | 100% |
| Casuarinin | 100% | 100% | 100% | 100% |
| 3-O-methylellegic acid | 98% | 97% | | |

Banaba extracts 1-4 were made as described below: the leave of banaba was first extracted with 25% acetone. After the acetone was evaporated, the extract was partitioned between ethyl acetate and water. The ethyl acetate was evaporated ant the extract was dried to make Banaba extract-1. The water extract was dried to be Banaba extract-2. Banaba extract-3 was made from using 90% ethanol/water and extracted at 40° C. on banaba leaves. After the ethanol alcohol was evaporated, the extract was dried to be Banaba extract-3. After 90% ethanol extraction, the banaba leaves were continuously extracted with water at 40° C. The water extract was dried to make Banaba extract-4. Foenugreek PE 20% contains 20% 4-hydroxyl isoleucine.

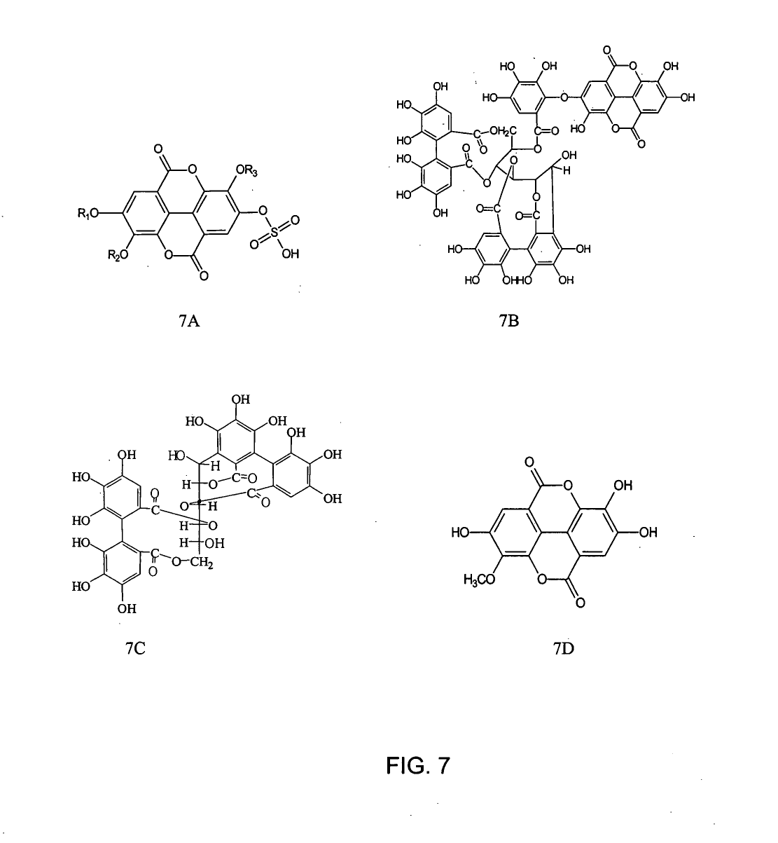

Banasulin, Lagerstroemin, Casuarinin, and 3-O-methylellegic acid (FIGS. 7A, 7B, 7C and 7D) were isolated from Banaba leaf from chromatography process. Their chemical structures were identified by spectroscopic method. Banasulin is a new compound having the structure as shown in FIG. 7A. Alternatively banasulin may have the following structure:

A compound of formula: 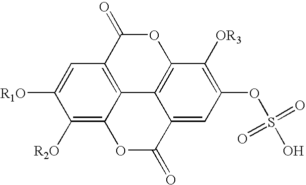
wherein:
R₁ is H, alkyl, alkenyl, alkynyl, benzene, sulfate, 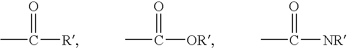
R′ is alkyl, alkenyl, alkynyl, benzene.
R₂ is H, alkyl, alkenyl, alkynyl, benzene, sulfate, 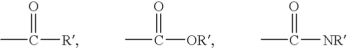
R′ is alkyl, alkenyl, alkynyl, benzene.
R₃ is H, alkyl, alkenyl, alkynyl, benzene, sulfate, 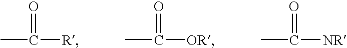
R′ is alkyl, alkenyl, alkynyl, benzene.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A composition of matter comprising 20% 4-hydroxyisolucine, banaba extract, huzhang extract, yerba mate extract, and green tea extract in an amount effective to promote body weight loss and/or loss of body fat in a subject.

2. The composition of claim 1, further comprising vitamin C and licorice extract.

3. The composition of claim 2, wherein said components are present in the following approximate ratios:

| | |
|---|---|
| 20% 4-Hydroxyisolucine | 6-14% |
| Yerba mate extract | 34-66% |
| Banaba extract | 16-21% |
| Green tea extract | 3-14% |
| Huzhang extract | 3-14% |
| Vitamin C | 0.3-0.7% |
| Licorice extract | 3-5% |

4. The composition of claim 2, wherein said components are present in the following approximate amounts:

| | | |
|---|---|---|
| 20% 4-Hydroxyisolucine | 20-200 | mg |
| Yerba mate extract | 200-500 | mg |
| Banaba extract | 50-300 | mg |
| Green tea extract | 10-200 | mg |
| Huzhang extract | 10-200 | mg |
| Vitamin C | 1-10 | mg |
| Licorice extract | 10-50 | mg |

5. The composition of claim 1, further comprising goat's rue.

6. The composition of claim 2, further comprising goat's rue.

7. The composition of claims 1, 2, 5 or 6, formulated as a tablet, capsule or pill.

8. A weight loss method, comprising administering to a subject a formulation comprising goat's rue, banaba extract, huzhang extract, yerba mate, and green tea extract in an amount effective to promote body weight loss and/or loss of body fat in said subject.

9. The weight loss method of claim 8, wherein said formulation further comprises vitamin C and licorice extract.

10. The weight loss method of claim 9, wherein said components are present in the following approximate ratios:

| | |
|---|---|
| Goat's rue extract | 6-14% |
| Yerba mate extract | 34-66% |
| Banaba extract | 16-21% |
| Green tea extract | 3-14% |
| Huzhang extract | 3-14% |
| Vitamin C | 0.0-0.7% |
| Licorice extract | 0-5% |

11. The weight loss method of claim 9, wherein said components are present in the following approximate amounts:

| | | |
|---|---|---|
| Goat's rue extract | 20-200 | mg |
| Yerba mate extract | 200-500 | mg |
| Banaba extract | 50-300 | mg |
| Green tea extract | 10-200 | mg |
| Huzhang extract | 10-200 | mg |
| Vitamin C | 1-10 | mg |
| Licorice extract | 10-50 | mg |

12. A method for reducing at least one metabolic parameter in a subject, comprising administering to a subject a formulation comprising 4-hydroxyisolucine, banaba extract, huzhang extract, yerba mate, and green tea extract in an amount effective to reduce said at least one metabolic parameter.

13. The method of claim 12, wherein said metabolic parameter is selected from the group consisting of blood glucose levels, blood triglyceride levels, and blood cholesterol levels.

14. The method of claim 12, wherein said formulation further comprises vitamin C and licorice extract.

15. The method of claim 14, wherein said components are present in the following approximate ratios:

| | |
|---|---|
| 20% 4-hydroxyisolucine | 6-14% |
| Yerba mate extract | 34-66% |
| Banaba extract | 16-21% |
| Green tea extract | 3-14% |
| Huzhang extract | 3-14% |
| Vitamin C | 0.3-0.7% |
| Licorice extract | 3-5% |

16. The method of claim 14, wherein said components are present in the following approximate amounts:

| | | |
|---|---|---|
| 20% 4-hydroxyisolucine | 20-200 | mg |
| Yerba mate extract | 200-500 | mg |
| Banaba extract | 50-300 | mg |
| Green tea extract | 10-200 | mg |
| Huzhang extract | 10-200 | mg |
| Vitamin C | 1-10 | mg |
| Licorice extract | 10-50 | mg |

17. The method of claim 12, wherein said formulation further comprises goat's rue.

18. The method of claim 14, wherein said formulation further comprises goat's rue.

19. The method of claim 12, wherein said formulation is in the form of a capsule, tablet, liquid or powder.

20. A method for decreasing the adipogenesis process comprising administering an effective amount of the composition according to claims 1, 2, 5 or 6 to a human or mammal in need thereof whereby said composition blocks fat cell synthesis by inhibiting undifferentiated 3T3-L1 cell to block fat synthesis.

21. A method for decreasing the adipogenesis process comprising administering an effective amount of Banaba extract to a human or mammal in need thereof whereby said composition blocks fat cell synthesis by inhibiting undifferentiated 3T3-LI cell to block fat synthesis.

22. A method for decreasing the adipogenesis process comprising administering an effective amount of Yerba mate extract to a human or mammal in need thereof whereby said composition blocks fat cell synthesis by inhibiting undifferentiated 3T3-L1 cell to block fat synthesis.

23. A method for decreasing the adipogenesis process comprising administering an effective amount of Foenugreek PE 20% to a human or mammal in need thereof whereby said composition blocks fat cell synthesis by inhibiting undifferentiated 3T3-L1 cell to block fat synthesis.

24. A method for decreasing the adipogenesis process comprising administering an effective amount of Banasulin to a human or mammal in need thereof whereby said composition blocks fat cell synthesis by inhibiting undifferentiated 3T3-L1 cell to block fat synthesis.

25. A method for decreasing the adipogenesis process comprising administering an effective amount of Lagerstroemin, Casuarinin, or 3-O-methylellegic acid to a human or mammal in need thereof whereby said composition blocks fat cell synthesis by inhibiting undifferentiated 3T3-L1 cell to block fat synthesis.

26. A method for reducing total cholesterol, triglycerides and LDL-cholesterol levels which comprise administering an effective amount of the composition according to claims 1, 2, 5 or 6 to a human or mammal in need thereof.

27. A method for reducing glucose levels which comprise administering an effective amount of the composition according to claims 1, 2, 5 or 6 to a human or mammal in need thereof.

28. A method for reducing total cholesterol, triglycerides and LDL-cholesterol levels which comprise administering an effective amount of Banaba extract to a human or mammal in need thereof.

29. A method for reducing glucose levels which comprise administering an effective amount of Banaba extract to a human or mammal in need thereof.

30. A method for reducing bodyweight, body fat, and triglyceride levels which comprise administering an effective amount of Yerba mate leaf extract to a human or mammal in need thereof.

31. A compound of formula: 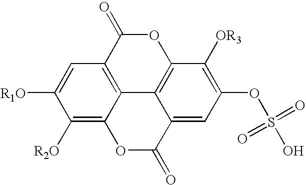
wherein:
R₁ is H, alkyl, alkenyl, alkynyl, benzene, sulfate, 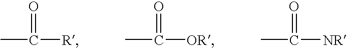
R′ is alkyl, alkenyl, alkynyl, benzene.
R₂ is H, alkyl, alkenyl, alkynyl, benzene, sulfate, 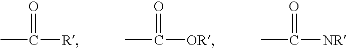
R′ is alkyl, alkenyl, alkynyl, benzene.
R₃ is H, alkyl, alkenyl, alkynyl, benzene, sulfate, 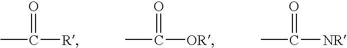
R′ is alkyl, alkenyl, alkynyl, benzene.

32. A compound of formula: [chemical structure]
